(19) United States
(12) Patent Application Publication
(10) Pub. No.: US 2008/0113983 A1
(43) Pub. Date: May 15, 2008

(54) Nicotinic Acetylcholine Receptor Ligands

(75) Inventors: Marc Chapdelaine, Wilmington, DE (US); Hui-Fang Chang, Wilmington, DE (US); Keith J. Herzog, Wilmington, DE (US); Carey Horchler, Wilmington, DE (US); Eifion Phillips, Bothwyn, PA (US)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: AstraZeneca AB, Sodertalje (SE)

(21) Appl. No.: 11/721,481

(22) PCT Filed: Dec. 13, 2005
(86) PCT No.: PCT/SE05/01909

§ 371 Date: Jun. 12, 2007

Related U.S. Application Data
(60) Provisional application No. 60/636,362, filed on Dec. 15, 2004.
(60) Provisional application No. 60/643,319, filed on Jan. 12, 2005.

(51) Int. Cl. A61K 31/5386; C07D 401/14; A61K 31/438; A61P 25/24; A61P 25/28; A61P 25/00; A61P 25/34; C07D 413/14

(52) U.S. Cl. 514/233.2; 546/18; 514/278; 544/70

(57) ABSTRACT

Acetylcholine receptor ligands of formula I wherein A, Ar¹ and Ar² are as described in the specification, diastereoisomers, enantiomers, pharmaceutically-acceptable salts, methods of making, pharmaceutical compositions containing and methods for using the same.

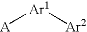

### FIELD OF THE INVENTION

This invention relates to oxazolidinones or pharmaceutically-acceptable salts thereof, processes for preparing them, pharmaceutical compositions containing them and their use in therapy. The invention also relates to compounds that are ligands for nicotinic acetylcholine receptors (nAChRs).

### BACKGROUND OF THE INVENTION

The use of compounds which bind nicotinic acetylcholine receptors in the treatment of a range of disorders involving reduced cholinergic function such as Alzheimer's disease, cognitive or attention disorders, anxiety, depression, smoking cessation, neuroprotection, schizophrenia, analgesia, Tourette's syndrome, and Parkinson's disease has been discussed in McDonald et al. (1995) “Nicotinic Acetylcholine Receptors: Molecular Biology, Chemistry and Pharmacology”, Chapter 5 in Annual Reports in Medicinal Chemistry, vol. 30, pp. 41-50, Academic Press Inc., San Diego, Calif.; and in Williams et al. (1994) “Neuronal Nicotinic Acetylcholine Receptors,” Drug News & Perspectives, vol. 7, pp. 205-223.

### SUMMARY OF THE INVENTION

This invention concerns nicotinic acetylcholine receptor-active compounds according to formula I:

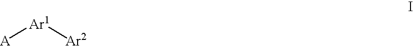

wherein:

A is

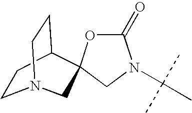

Ar¹ and Ar² are independently a 5- or 6-membered aromatic or heteroaromatic moiety having 0, 1 or 2 nitrogen atoms, 0 or 1 oxygen atoms, and 0 or 1 sulfur atoms;

wherein Ar¹ is unsubstituted or has 1, 2 or 3 substituents independently selected from -C₁-C₆alkyl, -C₂-C₆alkenyl, -C₂-C₆alkynyl, —CN, —NO₂, —CF₃, —S(O)ₙR¹ where n is 0, 1 or 2, —NR¹R², —CH₂—NR¹R², —OR², —CH₂OR² or —CO₂R², where at each occurrence R¹ and R² are independently selected from hydrogen or -C₁-C₆alkyl, or —NR¹R² in combination is —(CH₂)ⱼG(CH₂)ₖ- wherein G is a bond, oxygen, sulfur or NR³ where R³ is selected from hydrogen, C₁₋₆alkyl, aryl or heteroaryl; j is 2, 3 or 4, and k is 0, 1 or 2, and

Ar² is unsubstituted or has 1, 2 or 3 substituents selected from —C(═O)—NR¹R² or —NR¹—C(═O)—R².

The invention also encompasses stereoisomers, enantiomers, in vivo-hydrolysable precursors and pharmaceutically-acceptable salts of compounds of formula I, pharmaceutical compositions and formulations containing them, methods of using them to treat diseases and conditions either alone or in combination with other therapeutically-active compounds or substances, processes and intermediates used to prepare them, uses of them as medicaments, uses of them in the manufacture of medicaments and uses of them for diagnostic and analytic purposes.

### DETAILED DESCRIPTION OF THE INVENTION

Compounds of the invention are those according to formula I:

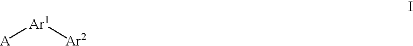

wherein:

A is

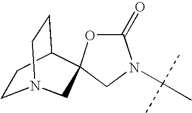

Ar¹ and Ar² are independently a 5- or 6-membered aromatic or heteroaromatic moiety having 0, 1 or 2 nitrogen atoms, 0 or 1 oxygen atoms, and 0 or 1 sulfur atoms;

wherein Ar¹ is unsubstituted or has 1, 2 or 3 substituents independently selected from -C₁-C₆alkyl, -C₂-C₆alkenyl, -C₂-C₆alkynyl, —CN, —NO₂, —CF₃, —S(O)ₙR¹ where n is 0, 1 or 2, —NR¹R², —CH₂—NR¹R², —OR², —CH₂OR or —CO₂R², where at each occurrence R¹ and R² are independently selected from hydrogen or -C₁-C₆alkyl, or —NR¹R² in combination is —(CH₂)ⱼG(CH₂)ₖ- wherein G is a bond, oxygen, sulfur or NR³ where R³ is selected from hydrogen, C₁₋₆alkyl, aryl or heteroaryl; j is 2, 3 or 4, and k is 0, 1 or 2, and Ar² is unsubstituted or has 1, 2 or 3 substituents selected from —C(═O)—NR¹R² or —NR¹—C(═O)—R²;

and stereoisomers, enantiomers, in vivo-hydrolysable precursors and pharmaceutically-acceptable salts thereof.

Particular compounds are those of formula I wherein: wherein:

Ar¹ is a 5-membered heteroaromatic ring having 1 oxygen atom or 1 sulfur atom and

Ar² is a 6-membered aromatic ring or a heteroaromatic ring having 0, 1 or 2 nitrogen atoms, 0 or 1 oxygen atoms, and 0 or 1 sulfur atoms.

More particular compounds are those of formula I wherein:

Ar¹ is an unsubstituted 5-membered heteroaromatic ring having 1 oxygen atom or 1 sulfur atom;

Ar² is a 6-membered aromatic ring or a heteroaromatic ring having 0, 1 or 2 nitrogen atoms, 0 or 1 oxygen atoms, and 0 or 1 sulfur atoms substituted with 1 or 2 substituents selected from —C(═O)—NR¹R² or —NR¹—C(═O)—R² where at each occurrence R¹ and R² are independently selected from hydrogen or -C₁-C₆alkyl, or —NR¹R² in combination is —(CH₂)ⱼG(CH₂)ₖ- wherein G is a bond, oxygen, sulfur or NR³ where R³ is selected from hydrogen, C₁₋₆alkyl, aryl or heteroaryl; j is 2, 3 or 4, and k is 0, 1 or 2.

Still more particular compounds are those of formula I wherein:

Ar¹is an unsubstituted 5-membered heteroaromatic ring having 1 oxygen atom or 1 sulfur atom;

Ar² is a 6-membered aromatic ring or a heteroaromatic ring having 0, 1 or 2 nitrogen atoms, 0 or 1 oxygen atoms, and 0 or 1 sulfur atoms substituted with 1 substituent selected from —C(═O)—NR¹R² or —NR¹—C(═O)—R² where at each occurrence R¹ and R² are independently selected from hydrogen or -C₁-C₆alkyl, or —NR¹R² in combination is —(CH₂)ⱼG(CH₂)ₖ- wherein G is a bond, oxygen, sulfur or NR³ where R³ is selected from hydrogen, C₁₋₆alkyl, aryl or heteroaryl; j is 2, 3 or 4, and k is 0, 1 or 2.

Other compounds of the invention wherein Ar¹ is D and Ar² is Ar are those according to formula Ia:

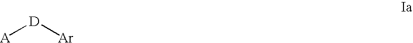

wherein:

A is as defined for formula I;

D a 5-membered heteroaromatic moiety having one nitrogen atom and one oxygen atom or a heteroaromatic moiety having one nitrogen atom and one sulfur atom, and

wherein Ar is unsubstituted or has 1, 2 or 3 substituents independently selected from -C₁-C₆alkyl, -C₂-C₆alkenyl, -C₂-C₆alkynyl, —CN, —NO₂, —CF₃, —S(O)ₙR¹ where n is 0, 1 or 2, —NR², —CH₂—NR¹R², —OR², —CH₂OR² or —CO₂R², where at each occurrence R¹ and R² are independently selected from hydrogen or -C₁-C₆alkyl, or —NR¹R² in combination is —(CH₂)ⱼG(CH₂)ₖ- wherein G is a bond, oxygen, sulfur or NR³ where R³ is selected from hydrogen, C₁₋₆alkyl, aryl or heteroaryl; j is 2, 3 or 4, and k is 0, 1 or 2;

and stereoisomers, enantiomers, in vivo-hydrolysable precursors and pharmaceutically-acceptable salts thereof.

Particular compounds are those of formula Ia wherein:

wherein:

D is selected from moieties according to formulae II, III, IV and V

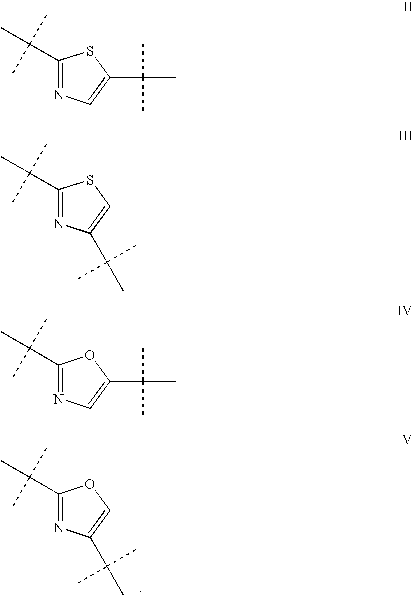

More particular compounds are those of formula Ia wherein:

Ar is a 6-membered aromatic ring or a heteroaromatic ring having 0, 1 or 2 nitrogen atoms, substituted with 1 or 2 substituents selected from —C(═O)—NR¹R² or —NR¹—C(═O)—R² where at each occurrence R¹ and R² are independently selected from hydrogen or -C₁-C₆alkyl, or —NR² in combination is —(CH₂)ⱼG(CH₂)ₖ- wherein G is a bond, oxygen, sulfur or NR³ where R³ is selected from hydrogen, C₁₋₆alkyl, aryl or heteroaryl; j is 2, 3 or 4, and k is 0, 1 or2.

Particular compounds of the invention are those described herein and pharmaceutically-acceptable salts thereof.

In a further aspect the invention encompasses compounds according to formula I or Ia wherein one or more of the atoms is a radioisotope of the same element. In a particular form of this aspect of the invention the compound of formula I or Ia is labeled with tritium. Such radio-labeled compounds are synthesized either by incorporating radio-labeled starting materials or, in the case of tritium, exchange of hydrogen for tritium by known methods. Known methods include (1) electrophilic halogenation, followed by reduction of the halogen in the presence of a tritium source, for example, by hydrogenation with tritium gas in the presence of a palladium catalyst, or (2) exchange of hydrogen for tritium performed in the presence of tritium gas and a suitable organometallic (e.g. palladium) catalyst.

Compounds of the invention labeled with tritium are useful for the discovery of novel medicinal compounds which bind to and modulate the activity, by agonism, partial agonism, or antagonism, of the α7 nicotinic acetylcholine receptor. Such tritium-labeled compounds may be used in assays that measure the displacement of a such compounds to assess the binding of ligand that bind to α7 nicotinic acetylcholine receptors.

In another aspect the invention relates to compounds according to formula I or Ia and their use in therapy and to compositions containing them.

In another aspect the invention encompasses the use of compounds according to formula I or Ia for the therapy of diseases mediated through the action of nicotinic acetylcholine receptors. A more particular aspect of the invention relates to the use of compounds of formula I or Ia for the therapy of diseases mediated through the action of α7 nicotinic acetylcholine receptors.

Another aspect of the invention encompasses a method of treatment or prophylaxis of diseases or conditions in which activation of the α7 nicotinic receptor is beneficial which method comprises administering a therapeutically-effective amount of a compound of the invention to a subject suffering from said disease or condition.

One embodiment of this aspect of the invention is a method of treatment or prophylaxis, wherein the disorder is anxiety, schizophrenia, mania or manic depression.

Another embodiment of this aspect of the invention is a method of treatment or prophylaxis of neurological disorders, psychotic disorders or intellectual impairment disorders, which comprises administering a therapeutically effective amount of a compound of the invention.

Another embodiment of this aspect of the invention is a method of treatment or prophylaxis, wherein the disorder is Alzheimer's disease, learning deficit, cognition deficit, attention deficit, memory loss, or Attention Deficit Hyperactivity Disorder.

Another embodiment of this aspect of the invention is a method of treatment or prophylaxis, wherein the disorder is Parkinson's disease, Huntington's disease, Tourette's syndrome, or neurodegenerative disorders in which there is loss of cholinergic synapses.

Another embodiment of this aspect of the invention is a method of treatment or prophylaxis of jetlag, nicotine addiction, craving, pain, and for ulcerative colitis, which comprises administering a therapeutically effective amount of a compound of the invention.

Yet another embodiment of this aspect of the invention is a method for inducing the cessation of smoking which comprises administering an effective amount of a compound of the invention.

Another embodiment of this aspect of the invention is a pharmaceutical composition comprising a compound of the invention and a pharmaceutically-acceptable diluent, lubricant or carrier.

A further aspect of the invention relates to a pharmaceutical composition useful for treating or preventing a condition or disorder mentioned herein arising from dysfunction of nicotinic acetylcholine receptor neurotransmission in a mammal, preferably a human, comprising an amount of a compound of formula I, an enantiomer thereof or a pharmaceutically-acceptable salt thereof, effective in treating or preventing such disorder or condition, and pharmaceutically-acceptable additives carrier.

Another embodiment of this aspect of the invention relates to use of a pharmaceutical composition of the invention for the treatment, amelioration or prophylaxis of human diseases or conditions in which activation of the α7 nicotinic receptor is beneficial.

Another embodiment of this aspect of the invention is the use of the pharmaceutical composition of the invention for the treatment or prophylaxis of neurological disorders, psychotic disorders or intellectual impairment disorders.

Another embodiment of this aspect of the invention is the use of the pharmaceutical composition of the invention for the treatment or prophylaxis of Alzheimer's disease, learning deficit, cognition deficit, attention deficit, memory loss, Attention Deficit Hyperactivity Disorder, anxiety, schizophrenia, or mania or manic depression, Parkinson's disease, Huntington's disease, Tourette's syndrome, neurodegenerative disorders in which there is loss of cholinergic synapse, jetlag, cessation of smoking, nicotine addiction including that resulting from exposure to products containing nicotine, craving, pain, and for ulcerative colitis.

A further aspect of the invention is the use of a compound according to the invention, an enantiomer thereof or a pharmaceutically-acceptable salt thereof, in the manufacture of a medicament for the treatment or prophylaxis of the diseases or conditions mentioned herein.

Another embodiment of this aspect of the invention is the use of a compound of the invention in the manufacture of a medicament for the treatment or prophylaxis of human diseases or conditions in which activation of the α7 nicotinic receptor is beneficial.

Another embodiment of this aspect of the invention is the use of a compound of the invention in the manufacture of a medicament for the treatment or prophylaxis of neurological disorders, psychotic disorders or intellectual impairment disorders.

Another embodiment of this aspect of the invention is the use of a compound of the invention in the manufacture of a medicament for treatment or prophylaxis of Alzheimer's disease, learning deficit, cognition deficit, attention deficit, memory loss or Attention Deficit Hyperactivity Disorder.

Another embodiment of this aspect of the invention is the use of a compound of the invention in the manufacture of a medicament for treatment or prophylaxis of anxiety, schizophrenia, or mania or manic depression.

Another embodiment of this aspect of the invention is the use of a compound of the invention in the manufacture of a medicament for treatment or prophylaxis of Parkinson's disease, Huntington's disease, Tourette's syndrome, or neurodegenerative disorders in which there is loss of cholinergic synapses.

Another embodiment of this aspect of the invention is the use of a compound as described above in the manufacture of a medicament for the treatment or prophylaxis of jetlag, pain, or ulcerative colitis.

Another aspect of the invention relates to the use of a compound of the invention in the manufacture of a medicament for facilitating the cessation of smoking or the treatment of nicotine addiction or craving including that resulting from exposure to products containing nicotine.

Another aspect of the invention relates to the use of a compound of the invention in combination with other therapeutically-active compounds or substances in pharmaceutical compositions or formulations, methods to treat diseases and conditions, uses as medicaments and uses in the manufacture of medicaments. Particular embodiments of this aspect of the invention comprise other therapeutically-active compounds or substances selected from sedatives, hypnotics, anxiolytics, antipsychotics, antianxiety agents, tranquilizers, and the like.

For the uses, methods, medicaments and pharmaceutical compositions mentioned herein the amount of compound used and the dosage administered will, of course, vary with the compound employed, the mode of administration and the treatment desired. However, in general, satisfactory results are obtained when the compounds of the invention are administered at a daily dosage of from about 0.1 mg to about 20 mg/kg of animal body weight. Such doses may be given in divided doses 1 to 4 times a day or in sustained release form. For man, the total daily dose is in the range of from 5 mg to 1,400 mg, more preferably from 10 mg to 100 mg, and unit dosage forms suitable for oral administration comprise from 2 mg to 1,400 mg of the compound admixed with a solid or liquid pharmaceutical carriers, lubricants and diluents.

The compounds of formula I, an enantiomer thereof, and pharmaceutically-acceptable salts thereof, may be used on their own or in the form of appropriate medicinal preparations for enteral or parenteral administration. According to a further aspect of the invention, there is provided a pharmaceutical composition including preferably less than 80% and more preferably less than 50% by weight of a compound of the invention in admixture with an inert pharmaceutically-acceptable diluent, lubricant or carrier.

Examples of diluents, lubricants and carriers are:

for tablets and dragees: lactose, starch, talc, stearic acid;

for capsules: tartaric acid or lactose;

for injectable solutions: water, alcohols, glycerin, vegetable oils;

for suppositories: natural or hardened oils or waxes.

There is also provided a process for the preparation of such a pharmaceutical composition which process comprises mixing the ingredients.

Compounds according to the invention are agonists of nicotinic acetylcholine receptors. While not being limited by theory, it is believed that agonists of the (α7 nicotinic acetylcholine receptor (nAChR) subtype are useful in the treatment or prophylaxis of neurological disorders, psychotic disorders and intellectual impairment disorders, and to have advantages over compounds which are or are also agonists of the (α4 nAChR subtype. Therefore, compounds which are selective for the 0:7 nAChR subtype are preferred. The compounds of the invention are indicated as pharmaceuticals, in particular in the treatment or prophylaxis of neurological disorders, psychotic disorders and intellectual impairment disorders. Examples of psychotic disorders include schizophrenia, mania and manic depression, and anxiety. Examples of intellectual impairment disorders include Alzheimer's disease, learning deficit, cognition deficit, attention deficit, memory loss, and Attention Deficit Hyperactivity Disorder. The compounds of the invention may also be useful as analgesics in the treatment of pain, chronic pain, and in the treatment or prophylaxis of Parkinson's disease, Huntington's disease, Tourette's syndrome, and neurodegenerative disorders in which there is loss of cholinergic synapses.

Compounds of the invention may further useful for the treatment or prophylaxis of jetlag, for use in inducing the cessation of smoking, craving, and for the treatment or prophylaxis of nicotine addiction including that resulting from exposure to products containing nicotine.

It is also believed that compounds according to the invention are useful in the treatment and prophylaxis of ulcerative colitis.

The compounds of the invention have the advantage that they may be less toxic, be more efficacious, be longer acting, have a broader range of activity, be more potent, produce fewer side effects, are more easily absorbed or have other useful pharmacological properties.

The compounds of formula I or Ia exist in tautomeric or enantiomeric forms, all of which are included within the scope of the invention. The various optical isomers may be isolated by separation of a racemic mixture of the compounds using conventional techniques, e.g. fractional crystallization, or chiral HPLC. Alternatively the individual enantiomers may be made by reaction of the appropriate optically active starting materials under reaction conditions which will not cause racemization.

As used herein, unless otherwise indicated, “C₁₋₄alkyl” includes but is not limited to methyl, ethyl, n-propyl, n-butyl, i-propyl, i-butyl, t-butyl, s-butyl moieties, whether alone or part of another group, C₁₋₄alkyl groups may be straight-chained or branched, and C₃₋₄ alkyl groups include the cyclic alkyl moieties cyclopropyl and cyclobutyl.

As used herein, unless otherwise indicated, “C₂₋₄alkenyl” includes but is not limited to 1-propenyl, 2-propenyl, 1-butenyl, 2-butenyl and 3-butenyl.

As used herein, unless otherwise indicated, “C₂₋₄alkynyl” includes but is not limited to ethynyl, 1-propynyl, 2-propynyl, 1-butynyl, 2-butynyl and 3-butynyl.

As used herein, unless otherwise indicated, aryl refers to a phenyl ring which may have 1, 2 or 3 substituents selected from: halogen, C₁₋₄alkyl, C₂₋₄alkenyl, C₂₋₄alkynyl, C₁₋₄alkyl, CN, NO₂, and CF₃ .

As used herein, unless otherwise indicated, heteroaryl refers to a 5- or 6-membered aromatic or heteroaromatic ring having 1, 2 or 3 heteroatoms selected from nitrogen oxygen and sulfur, provided that heteroaromatic rings contains at least one nitrogen, oxygen, or sulfur atom.

As used herein, unless otherwise indicated, halogen refers to fluorine, chlorine, bromine, or iodine.

Where necessary, hydroxy, amino, or other reactive groups may be protected using a protecting group as described in the standard text “Protecting groups in Organic Synthesis”, 3^{rd} Edition (1999) by Greene and Wuts.

Unless otherwise stated, reactions are conducted under an inert atmosphere, preferably under a nitrogen atmosphere and are usually conducted at a pressure of about one to about three atmospheres, preferably at ambient pressure (about one atmosphere).

The compounds of the invention and intermediates may be isolated from their reaction mixtures by standard techniques.

Acid addition salts of the compounds of formula I or Ia which may be mentioned include salts of mineral acids, for example the hydrochloride and hydrobromide salts; and salts formed with organic acids such as formate, acetate, maleate, benzoate, tartrate, and fumarate salts.

Acid addition salts of compounds of formula I or Ia may be formed by reacting the free base or a salt, enantiomer or protected derivative thereof, with one or more equivalents of the appropriate acid. The reaction may be carried out in a solvent or medium in which the salt is insoluble or in a solvent in which the salt is soluble, e.g., water, dioxane, ethanol, tetrahydrofuran or diethyl ether, or a mixture of solvents, which may be removed in vacuum or by freeze drying. The reaction may be a metathetical process or it may be carried out on an ion exchange resin.

### Pharmacology

The pharmacological activity of compounds of the invention may be measured by using tests such as those set out below:

### Assay for affinity at an α₇ nAChR receptor by measuring the binding of ¹²⁵I-α-bungarotoxin (BTX) binding to rat hipiocampal membranes.

Rat brain cell membranes bearing α₇ nAChR receptors may be prepared by homogenizing hippocampus tissue in 20 volumes of cold homogenization buffer (HB): mM concentrations of HB constituents: tris(hydroxymethyl)aminomethane 50; CaCl₂ 2; MgCl₂ 1; NaCl 120; KCl 5: pH 7.4). Homogenates are centrifuged for 5 minutes at 1000×g, the supernatant saved and the pellets re-extracted and centrifuged. Pooled supernatants are and centrifuged for 20 minutes at 12000×g, the pelleted membranous material is washed, and re-suspended in HB. Membranes (30-150 μg) are incubated with 3 nM [¹²⁵I]α-BTX, 1 mg/mL bovine serum albumin (BSA), together with test compounds in HB for 2 hours at room temperature with gentle shaking. Membranes may then be trapped on Whatman glass fiber filters (thickness C or B) using a Brandel cell harvester and washed 4 times. Pre-treating the filters for 3 hours with 1% (BSA/0.01% PEI (polyethyleneimine) in water will yield low filter blanks (0.07% of total counts per minute). Non-specific binding may be determined by 100 μM (-)-nicotine. Typically specific binding is about 75%.

### Assay for affinity at human α7 nAChR receptor by measuring the binding of ¹²⁵-α-bungarotoxin (BTX) binding to membranes.

Membranes may be prepared from cultured cells expressing human α₇ receptors by isolating a 500-40000×g membrane fraction. Such membranes may be used as described for rat brain membranes to assess the binding of compounds to human α₇ receptors.

### Assay for affinity at an α₄ nAChR receptor by measuring the binding of [³H]-(-)-nicotine binding to rat cortical membranes.

A procedure modified from Martino-Barrows and Kellar (Mol Pharm (1987) 31:169-174) may be used. Rat cortical brain membranes are prepared as described in the [¹²⁵I]α-BTX binding assay, except that a 500-12000×g membrane fraction is prepared. Membranes (30-150 μg) are incubated with 30-100 pM of the epibatidine analog [¹²⁵I]-IPH ((+/−)-exo-2-(2-iodo-5-pyridyl)-7-azabicyclo-[2,2,1]heptane) together with test compound in HB for 1 hour at room temperature with gentle shaking. Membranes may be recovered as described for the [¹²⁵I]α-BTX binding assay. Non-specific binding may be determined by 100 μM carbachol. Typically specific binding is about 84%.

### Analysis of Binding Data Obtained in α7 nAChR or α4 nAChR receptor assays

IC₅₀ values and pseudo Hill coefficients (nH) may be calculated using the non-linear curve fitting program ALLFIT (DeLean A, Munson P J and Rodbard D (1977) Am. J. Physiol., 235:E97-E102). Saturation curves may be fitted to a one site model, using the non-linear regression program ENZFITTER (Leatherbarrow, R. J. (1987)), yielding a Kd value for [¹²⁵I]-α-BTX binding to rat α7 nAChR of 1.7 nM and a Kd value for [¹²⁵I]-IPH binding to the rat α4 nAChR of 64 pM. Kᵢ values may be estimated using the general Cheng-Prusoff equation:

Kᵢ=[IC_{50 ]/((}2+([ligand]/K_{D})ⁿ)^{1/n}−1)

where a value of n=1 is used whenever n_{H}<1.5 and a value of n=2 is used when n_{H}>1.5. To account for variability, assays may be performed in triplicate and variability will typically be ±5%. Kᵢ values may be determined using six to 11 drug concentrations.

Compounds of the invention expected to have useful therapeutic activity will be found to have binding affinities (Kᵢ) of less than 10 μM in α7 nAChR receptor or α₄ nAChR receptor assays.

The compounds of the invention have the advantage that they may be less toxic, be more efficacious, be longer acting, have a broader range of activity, be more potent, produce fewer side effects, are more easily absorbed or have other useful pharmacological properties.

### Experimental Methods

The invention will now be illustrated by the following Examples in which, generally:

(i) operations are carried out at ambient temperature, i.e. in the range 17 to 25 ° C. and under an atmosphere of an inert gas such as argon or nitrogen unless otherwise stated;

(ii) evaporations are carried out by rotary evaporation in vacuo and work-up procedures are carried out after removal of residual solids by filtration;

(iii) column chromatography (by the flash procedure) and medium pressure liquid chromatography (MPLC) are performed on ICN Ecochrom 60 Angstrom silica gel. In cases where Reverse Phase High Pressure Liquid Chromatography (RP-HPLC) is employed as a method of purification, Gilson instrumentation (215 Injector, 333 Pumps and 155 UV/Vis Detector) and a Varian C8 reverse phase column (60 Angstrom irregular load in 8 μm particle size, 41.4 mm ID×250 mm) may be employed. Gradient elution may be performed with aqueous 0.1% trifluoroacetic acid /acetonitrile with 0.1% trifluoroacetic acid. Sample collection is based on signal at 254 Dm unless otherwise noted. In cases where Normal Phase High Pressure Liquid Chromatography (NP-HPLC) is required, Dynamax instrumentation (Dual SD-1 Pumps and UV-1 UV/is Detector with a Superprep Flow Cell and a Rainin silica normal phase column (60 Angstrom irregular load in 8 μm particle size, 41.4 mm ID×250 mm) may be employed. Isocratic elution may be performed with 0.5% isopropyl alcohol in hexanes. Supercritical Fluid Chromatography (SFC) may be performed on a Berger Autoprep SFC system generally using methanol (containing 0.5% dimethyl ethyl amine) in carbon dioxide and a Berger Diol column (5 micron, 60 Å pore size).

(iv) yields, where present, are not necessarily the maximum attainable;

(v) in general, the structures of the end-products of the Formula I or Ia are confirmed by nuclear magnetic resonance (NMR) and/or mass spectral (MS) techniques; AP/CI mass spectral data are obtained using a Waters Platform LCZ spectrometer and, where appropriate, either positive ion data or negative ion data are collected; NMR chemical shift values are measured on the delta scale proton magnetic resonance spectra are determined using a Bruker Avance 300 spectrometer operating at a field strength of 300 MHz; the following abbreviations have been used: s, singlet; d, doublet; t, triplet; q, quartet; m, multiplet; br, broad;

(vi) intermediates are not necessarily fully purified but their structures and purity are assessed by thin layer chromatographic, HPLC, infra-red (IR) and/or NMR analysis;

(vii) melting points are uncorrected and are determined using a Meltemp 3.0 melting point apparatus or an oil-bath apparatus; melting points for the end-products of the Formula I or Ia are determined after crystallization from an appropriate organic solvent or solvent mixture;

(viii) the following abbreviations have been used:

| | |
|---|---|
| DMF | N,N-dimethylformamide |
| DMSO | dimethylsulphoxide |
| THF | tetrahydrofuran |
| DMA | N,N-dimethylacetamide |
| DCM | dichloromethane |

### Preparation of Starting Materials:

Certain starting materials for the examples herein are either commercially available or are readily prepared by standard methods from known materials. The following reactions illustrate the preparation of particular intermediates.

### Intermediate A: 4-(5-Bromo-thiophen-2-yl)-N,N-dimethyl-benzamide

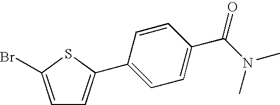

4-(N,N-Dimethylaminocarbonyl)phenylboronic acid (415 mg, 2.15 mmole), 2-5-dibromothiophene (1.14 grams, 4.73 mmole), cesium carbonate (2.1 grams, 6.45 mmole), and tetrakis(triphenylphosphine)palladium (240 mg, 0.22 mmole) were slurried in ethylene glycol dimethyl ether/water/ethanol (7:3:2, 20 mL). The mixture was heated in a round bottom flask at 80° C. overnight. The mixture was cooled, treated with water and extracted with chloroform (3 times). The organic layers were combined, dried over sodium sulfate, filtered, and evaporated under reduced pressure to afford the product as an oil. The material was purified on silica gel using 40% ethyl acetate in hexanes as the eluant. The compound was obtained as a pale yellow solid (59% recovery). ¹H NMR (300.132 MHz, DMSO) δ7.67 (d, J=9.4 Hz, 1H), 7.46-7.43 (m, 3H), 7.29 (d, J=4.7 Hz, 1H), 2.95 (s, 6H); MS m/z: 311 (M+H)⁺.

### Intermediate B: N-[3-(5-Bromo-thiophen-2-yl)-phenyl]-acetamide

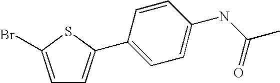

The title compound was prepared by a method analogous to that described for Intermediate A using 3-acetamidobenezeneboronic acid and 2-5-dibromothiophene. The compound was obtained as a tan solid (53% recovery). MS m/z: 299 (M+H)^{+.}

### Intermediate C: N-[3-(5-Bromo-thiophen-2-yl)-phenyl]-N-methyl-acetamide

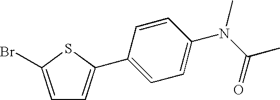

Intermediate B, N-[3-(5-Bromo-thiophen-2-yl)-phenyl]-acetamide, (93 mg, 0.31 mmole) was dissolved in N,N-dimethylformamide (8 mL) and stirred at 0° C. Sodium hydride (9 mg, 0.37 mmole) was added in one portion followed immediately by methyl iodide (53 mg, 0.37 mmole). The reaction course was followed by HPLC and determined to be incomplete after 1 hour. Additional methyl iodide (53 mg, 0.37 mmole) was added. The reaction course was followed by HPLC and determined to be complete after an additional 15 minutes at 0° C. The reaction mixture was treated with water and extracted with chloroform (3 times). The material was purified on silica gel using 40% ethyl acetate in hexanes as the eluant. The compound was obtained as a pale yellow solid (94% recovery). ¹H NMR (300.132 MHz, DMSO) δ7.65-7.42 (m, 4H), 7.31-7.26 (m, 2H), 3.32 (s, 3H), 3.19 (s, 3H); MS m/z: 311 (M+H)⁺.

### Intermediate D: N-[3-(5-Bromo-thiophen-2-yl)-phenyl]-propionamide

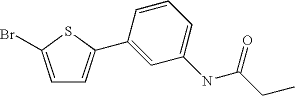

3-(5-Bromo-thiophen-2-yl)-phenylamine (168 mg, 0.68 mmole), was obtained in an analogous fashion to intermediate A, and dissolved in tetrahydrofuran (10 ). Proprionyl chloride (73 mg, 0.792 mmole) was added in one portion and the mixture was allowed to stir for 30 minutes at ambient temperature. Aqueous sodium hydroxide (1 N) was added the mixture was extracted with chloroform (3 times). The organic layers were combined, dried over sodium sulfate, filtered, and evaporated under reduced pressure to afford the product as an oil. The material was purified on silica gel using 40% ethyl acetate in hexanes as the eluant. The compound was obtained as a pale yellow solid (95% recovery). ¹H NMR (300.132 MHz, DMSO) δ9.95 (s, 1H), 7.88 (s, 1H), 7.53 (d, J=7.8 Hz, 1H), 7.37-7.24 (m, 4H), 2.33 (q, J=7.6 Hz, 2H), 1.09 (t, J=8.2 Hz, 3H).

### Intermediate E: 3′-(Thiazol-2-yl)spiro[1-azabicyclo[2.2.2]octan-3,5′-oxazolidin]-2′-one

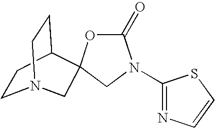

(a) Methyl N-(thiazol-2-yl)carbamate

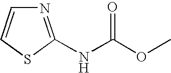

2-Aminothiazole (10.00 g, 99.86 mmole), triethylamine (20.2 g, 200 mmol) and a catalytic amount of 4-(N,N-dimethylamino)pyridine in anhydrous tetrahydrofuran (200 mL) were stirred at 0° C. Methyl chloroformate (18.9 g, 200 mmol) was added slowly to the mixture at 0 ° C. Then the reaction mixture was stirred at room temperature for several hours. The tetrahydrofuran was evaporated, the residue was dissolved in chloroform, and the resulting solution was washed with water, dried (MgSO₄), filtered, and then the solvent was evaporated. The residue was purified by flash chromatography using a gradient of ammoniated methanol in chloroform to give methyl N-(thiazol-2-yl)carbamate as a pale yellow solid (17.0 g), m/z 159 (MH⁺).

(b) 3′-(Thiazol-2-yl spiro[1-azabicyclo[2.2.2]octan-3,5′-oxazolidin]-2′-one

Spiro[1-azabicyclo[2.2.2]octan-3,2′-oxirane]-N1-trihydroboron (3.00 g, 19.6 mmol), methyl N-(thiazol-2-yl)-carbamate (3.90 g, 23.5 mmol) and tetra-n-butylammonium chloride (1.09 g, 3.92 mmol) were suspended in water (30 mL), and the reaction mixture was stirred at 80° C. overnight. After cooling to room temperature the reaction mixture was filtered, and the collected solid was washed with a small amount of water. The solid was then dissolved in acetone (60 mL), and 6 mL of concentrated hydrochloric acid was added. The mixture was stirred at room temperature overnight. The solvent was evaporated from the mixture, then the residue was partitioned between aqueous potassium carbonate and chloroform. The chloroform layer was dried (MgSO₄), filtered and evaporated to give title compound as a solid (3.30 g), m/z 266 (MH⁺).

### Intermediate F: 3′-(5-Bromothiazol-2-yl)spiro[1-azabicyclo[2.2.2]octan-3,5′-oxazolidin]-2′-one

### Method I

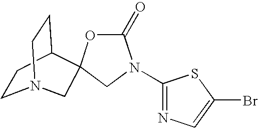

A solution containing 3′-(thiazol-2-yl)spiro[1-azabicyclo[2.2.2]octan -3,5′-oxazolidin]-2′-one (2.80 g, 10.55 mmol) and N-bromosuccinimide (3.90 g, 21.86 mmole) in N,N-dimethylformamide (15 mL) were stirred at RT overnight. The mixture was diluted with chloroform, washed with saturated aqueous potassium carbonate and then with brine, dried (MgSO₄), and then the solvent was evaporated. The residue was purified by flash chromatography using a gradient of ammoniated methanol in chloroform to give the title compound as a solid (1.70 g), m/z 343, 345 (M⁺).

### Method II

(a) Methyl N-(5-bromothiazol-2-yl)carbamate

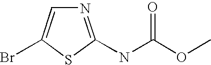

The title compound was prepared by a method analogous to that in Preparation 1(a) from 2-amino-5-bromothiazole. The title compound (4.10 g) was obtained as a yellow solid, m/z 237, 239 (MH⁺).

(b) 3′-(5-Bromothiazol-2-yl)spiro[1-azabicyclo[2.2.2]octan-3,5′-oxazolidin]-2′-one

The title compound was prepared by a method analogous to that described in Preparation 1(b) from spiro[1-azabicyclo[2.2.2]octan-3,2′-oxirane]-N1-trihydroboron and methyl N-(5-bromothiazol-2-yl)carbamate. The title compound (650 mg) was obtained as a solid, m/z 344, 346 (MH⁺).

The title compound was separated into its enantiomers by chiral supercritical fluid chromatography performed on a Chiral Pak AS-H Column using 1:1 isopropanol and supercritical carbon dioxide containing 0.5% dimethyl ethyl amine as the eluant to give the title compounds as colorless solids each with m/z 343 (MH⁺).

### Intermediate G: 3R)-3′-(5-Bromothiazol-2-yl)spiro[1-azabicyclo[2.2.2]octan-3,5′-oxazolidin-2′-one]

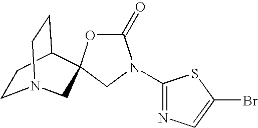

Sodium hydride (60% suspension in mineral oil) (15 mg, 0.36 mmol) was added to a solution of (3S)-spiro[1-azabicyclo[2.2.2]octan-3,5′-oxazolidin-2′-one] (55 mg, 0.30 mmol) in anhydrous N,N-dimethylformamide (0.8 mL), and the resulting mixture was stirred for 30 min at 50° C. 2,5-Dibromothiazole (87 mg, 0.36 mmole) was added into the reaction mixture, which was then stirred at 50° C. overnight. The solution was allowed to cool to room temperature, and then saturated aqueous ammonium chloride was added. The resulting mixture was extracted with chloroform, and the chloroform layer was washed with saturated aqueous potassium carbonate, then dried (MgSO₄), filtered, and evaporated. The residue was purified by flash chromatography using a gradient of ammoniated methanol in chloroform to give the title compound as a yellow oil (11 mg); m/z 344, 346(MH⁺).

### Intermediate H: (3R)-3′-(5-Bromo-thiazol-2-yl)spiro[1-azabicyclo[2.2.2]octan-3,5′-oxazolidin-2′-one] and (3S)-3′-(5-bromo-thiazol-2-ylspiro[1-azabicyclo[2.2.2]octan-3,5′-oxazolidin-2′-one]

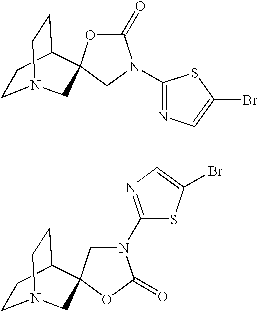

The title compounds (500 mg for each compound) were separating the racemic compound prepared in Preparation 2 by Supercritical Fluid Chromatography on a Chiral Pak AS-H colurnn (250×19 mm, 5L micro) using 12% methanol and 88% supercritical fluid carbon dioxide with 0.5% dimethyl ethyl amine additive as the eluant which give two off-white solids, each with m/z 344, 346 (MH⁺).

### EXAMPLE 1

### 4-[5-((R)-5-1-Aza-bicyclo[2.2.2]oct-3-yl-2-oxo-oxazolidin-3-yl)-thiophen-2-y1]-N,N-dimethyl-benzamide

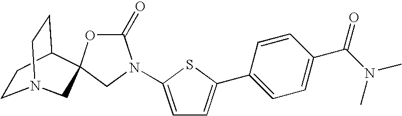

(3S)-Spiro[1-azabicyclo[2.2.2]octane-3,5′-oxazolidin]-2′-one (100 mg, 0.55 mmole), 4-(5-bromo-thiophen-2-yl)-N,N-dimethyl-benzamide (Intermediate A, 310 mg, 0.66 mmole), copper iodide (209 mg, 1.10 mmole), potassium carbonate (152 mg, 1.10 mmole), and ethane-1,2-diamine (66mg, 1.10 mmole) were combined in a reaction vial and slurried with 1,4-dioxane (2 mL). The reaction vial was sealed and heated at 110° C. overnight. The volatiles were removed under reduced pressure. The residue was taken up in methanol and run through a C18 reverse phase plug of silica gel. The resultant residue was then purified by silica gel using 5% 7 N ammoniated methanol in chloroform. The compound was obtained as a tan solid (47% recovery). ¹H NMR (300.132 MHz, DMSO) δ7.64 (d, J=7.9 Hz, 2H), 7.43-7.40 (m, 3H), 6.61 (d, J=4.1 Hz, 1H), 4.25 and 3.98 (AB, J=9 Hz, 2H), 3.05-2.92 (m, 7H), 2.81-2.64 (m, 3H), 2.10 (s, 1H), 1.85 (s, 2H), 1.72-1.54 (m, 2H), 1.55-1.40 (m, 2H); MS m/z: 412 (M+H)⁺.

### EXAMPLE 2

### 3-[5-((R)-5-1-Aza-bicyclo[2.2.2]oct-3-yl-2-oxo-oxazolidin-3-yl)-thiophen-2-yl]-N,N-dimethyl-benzamide

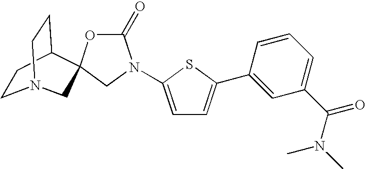

The title compound obtained as a tan solid in 22% yield using a process analogous to that of Example 1 and a thiophene intermediate prepared in a manner analogous to intermediate A. ¹H NMR (300.132 MHz, DMSO) δ7.64 (d, J=7.5 Hz, 1H), 7.57 (s, 1H), 7.46-7.39 (m, 2H), 7.25 (d, J=9.3 Hz, 1H), 6.60 (d, J=4.1 Hz, 1H), 4.24 and 3.98 (AB, J=11.0 Hz, 2H), 3.09-2.90 (m, 8H), 2.81-2.66 (m, 2H), 2.13-2.07 (m, 1H), 1.91-1.78 (m, 2H), 1.70-1.58 (m, 2H), 1.54-1.42 (m, 2H); MS m/z: 412 (M+H)+.

### EXAMPLE 3

### N-{3-[5-((R)-5-1-Aza-bicyclo[2.2.2]oct-3-yl-2-oxo-oxazolidin-3-yl)-thiophen- 2-yl]-phenyl}-acetamide

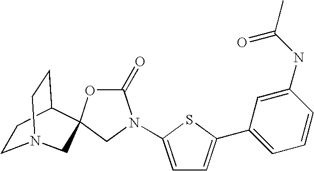

The title compound obtained as a tan solid in 35% yield using a process analogous to that of Example 1 with a thiophene intermediate obtained by a process analogous to that used to prepare Intermediate B. ¹H NMR (300.132 MHz, DMSO) δ9.96 (s, 1H), 7.84 (s, 1H), 7.48-7.42 (m, 2H), 7.31-7.22 (m, 2H), 6.58 (d, J=4.1 Hz, 1H), 4.23 and 3.97 (AB, J=10.0 Hz, 2H), 3.05 (s, 3H), 2.80-2.63 (m, 4H), 2.13-2.07 (m, 1H), 1.93-1.77 (m, 1H), 1.71-1.58 (m, 2H), 1.57-1.40 (m, 2H); MS m/z: 398 (M+H)+.

### EXAMPLE 4

### N-{4-[5-((R)-5-1-Aza-bicyclo[2.2.2]oct-3-yl-2-oxo-oxazolidin-3-yl)-thiophen-2-yl]-phenyl} -acetamide

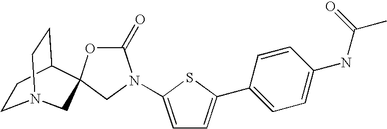

The title compound obtained as a tan solid in 28% yield using Intermediate B and a process analogous to that of Example 1. ¹H NMR (300.132 MHz, DMSO) δ9.97 (s, 1H), 7.59 (d, J=8.7 Hz, 2H), 7.50 (d, J=8.6 Hz, 2H), 7.21 (d, J=3.9 Hz, 1H), 6.56 (d, J=3.9 Hz, 1H), 4.09 (AB, J=10.6 Hz, 2H), 3.06 (s, 2H), 2.81-2.63 (m, 4H), 2.10 (s, 1H), 2.04 (s, 3H), 1.90-1.77 (m, 1H), 1.69-1.56 (m, 2H), 1.54-1.40 (m, 1H); MS m/z: 398 (M+H)+.

### EXAMPLE 5

### 5-(R)-1-Aza-bicyclo[2.2.2]oct-3-y1-3-{5-[3-(-pyrrolidine-1-carbonyl)-phenyl]-thiophen-2-y1}-oxazolidin-2-one

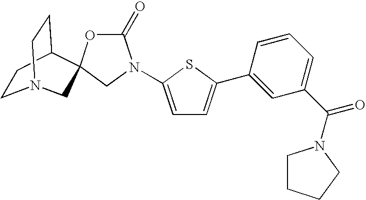

The title compound obtained as a white solid in 51% yield using a process analogous to that of Example 1 with a thiophene intermediate prepared by reacting 2,5-dibromothiophene with a commercially available boronic acid in manner analogous to the preparation of intermediate A. ¹H NMR (300.132 MHz, DMSO) δ7.68-7.63 (m, 2H), 7.47 -7.34 (m, 3H), 6.60 (d, J=4.1 Hz, 1H), 4.24 and 3.98 (AB, J=9.5 Hz, 2H), 3.51-3.35 (m, 4H), 3.10-3.02 (m, 1H), 2.81-2.65 (m, 3H), 2.11 (s, 1H), 1.93-1.77 (m, 5H), 1.71-1.56 (m, 2H), 1.54-1.40 (m, 1H); MS m/z: 438 (M+H)+.

### EXAMPLE 6

### N-{3-[5-((R)-5-1-Aza-bicyclo[2.2.2]oct-3-yl-2-oxo-oxazolidin-3-yl)-thiophen-2-yl]-phenyl}-propionamide

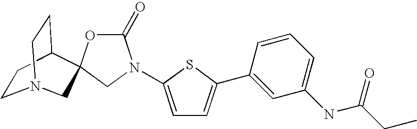

The title compound obtained as a white solid in 47% yield by a process analogous to that of Example 1 with a thiophene intermediate obtained by a process analogous to that used to prepare Intermediate D. ¹H NMR (300.132 MHz, DMSO) δ9.88 (s, 1H), 7.88 (s, 1H), 7.49-7.44 (m, 1H), 7.31-7.24 (m, 3H), 6.58 (d, J=4.3 Hz, 1H), 4.24 and 3.97 (AB, J=10.0 Hz, 2H), 3.10-3.03 (m, 1H), 2.83-2.62 (m, 2H), 2.33 (q, J=-7.5 Hz, 4H), 2.11 (s, 1H), 1.92-1.79 (m, 2H), 1.69-1.57 (m, 2H), 1.54-1.43 (m, 1H), 1.09 (t, J-=8.5 Hz, 3H); MSm/z: 412(M+H)+.

### EXAMPLE 7

### N-{3-[5-((R)-5-1-Aza-bicyclo[2.2.2]oct-3-yl-2-oxo-oxazolidin-3-yl)-thiophen-2-yl]-phenyl}-N-methyl-acetamide

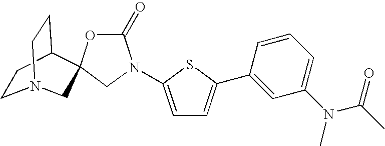

The title compound obtained as a white solid in 50% yield using a process analogous to that of Example 1 with a thiophene intermediate obtained by a process analogous to that used to prepare Intermediate C. ¹H NMR (300.132 MHz, DMSO) δ7.59-7.40 (m, 4H), 7.20 (d, J=8.5 Hz, 1H), 6.60 (d, J=4.1 Hz, 1H), 4.24 and 3.97 (AB, J=10.0 Hz, 2H), 3.19 (s, 4H), 3.05 (s, 3H), 2.81-2.65 (m, 4H), 2.11-2.06 (m, 1H), 1.90-1.79 (m, 2H), 1.70-1.57 (m, 2H), 1.52-1.43 (m, 1H); MS m/z: 412 (M+H)+.

### EXAMPLE 8

### N-{3-[5-((R)-5-1-Aza-bicyclo[2.2.2]oct-3-yl-2-oxo-oxazolidin-3-yl)-thiophen-2-yl]-phenyl} -N-methyl-propionamide

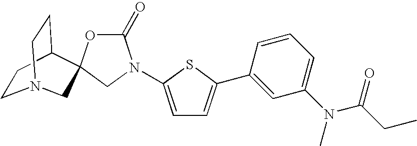

The title compound obtained as a white solid in 56% yield using a process analogous to that of Example 1 and intermediate D. ¹H NMR (300.132 MHz, DMSO) 6 7.57-7.40 (m, 4H), 7.18 (d, J=8.3 Hz, 1H), 6.60 (d, J=4.0 Hz, 1H), 4.24 and 3.19 (AB, J=9.5 Hz, 2H), (s, 3H), 3.06 (s, 2H), 2.80-2.64 (m, 4H), 2.13-2.04 (m, 3H), 1.92-1.78 (m, 2H), 1.71-1.56 (m, 2H), 1:53-1.41 (m, 1H), 0.94 (t, J=7.4 Hz, 3H); MS m/z: 426 (M+H)+.

### EXAMPLE 9

### N-{4-[5-((R)-5-1-Aza-bicyclo[2.2.2]oct-3-yl-2-oxo-oxazolidin-3-yl)-thiophen-2-yl]-phenyl}-N-methyl-acetamide

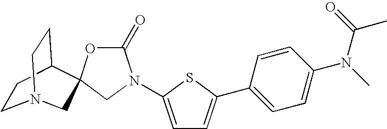

The title compound obtained as a white solid in 58% yield using Intermediate C and a process analogous to that of Example 1. ¹H NMR (300.132 MHz, DMSO) δ7.64 (d, J=8.1 Hz, 2H), 7.37-7.31 (m, 3H), 6.60 (d, J=4.2 Hz, 1H), 4.24 and 3.97 (AB, J=9.5 Hz, 2H), 3.16 (s, 3H), 3.05 (s, 2H), 2.80-2.64 (m, 4H), 2.10 (s, 1H), 1.91-1.75 (m, 3H), 1.73-1.56 (m, 2H), 1.57-1.41 (m, 2H); MS m/z: 412 (M+H)+.

### EXAMPLE 10

### 5-(R)-1-Aza-bicyclo[2.2.2]oct-3-yl-3- {5-[3-(morpholine-4-carbonyl)-phenyl]-thiophen-2-yl} -oxazolidin-2-one

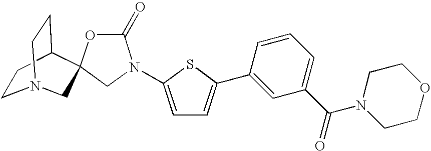

The title compound obtained as a white solid in 68% yield using a process analogous to that of Example 1 and an intermediate prepared by reacting 2,5-dibromothiophene with commercially available boronic acid in manner analogous to the preparation of intermediate A. ¹H NMR (300.132 MHz, DMSO) 6 7.68-7.57 (m, 2H), 7.48-7.40 (m, 2H), 7.26 (d, J=9.8 Hz, 1H), 6.61 (d, J=3.7 Hz, 1H), 4.24 and 3.98 (d, J=9.3 Hz, 2H), 3.70-3.36 (m, 8H), 2.80-2.66 (m, 4H), 2.14-2.08 (m, 1H), 1.93-1.76 (m, 2H), 1.75-1.55 (m, 2H), 1.56-1.41 (m, 2H); MS m/z: 454 (M+H)+.

### EXAMPLE 11

### N-{3-[5-((?)-5-1-Aza-bicyclo[2.2.2]oct-3-yl-2-oxo-oxazolidin-3-yl)-furan-2-yl]-phenyl}-propionamide

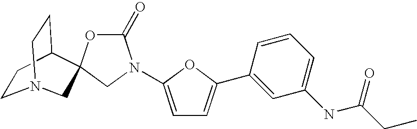

The title compound obtained as a white solid in 75% yield by a process analogous to that of Example 1 using an intermediate prepared using 2,5-dibromofaran in a manner analogous to that used for intermediate D. ¹H NMR (300.132 MHz, DMSO) δ9.91 (s, 1H), 7.83 (s, 1H), 7.54-7.50 (m, 1H), 7.36-7.29 (m, 2H), 6.87 (d, J=3.6 Hz, 1H), 6.26 (d, J=3.4 Hz, 1H), 4.24 and 4.02 (d, J=9.1 Hz, 2H), 3.05 (s, 2H), 2.79-2.64 (m, 4H), 2.33 (q, J=7.5 Hz, 2H), 2.12 (s, 1H), 1.90-1.79 (m, 1H), 1.67-1.59 (m, 2H), 1.53-1.42 (m, 1H), 1.09 (t, J=7.5 Hz, 3H); MS m/z: 396 (M+H)+.

### EXAMPLE 12

### 5-(R)-1-Aza-bicyclo[2.2.2]oct-3-yl-3-{5-[3-(pyrrolidine-1-carbonyl)-phenyl]- furan-2-yl}-oxazolidin-2-one

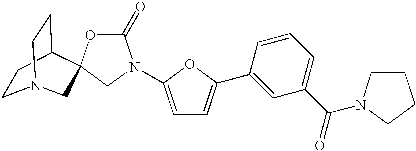

The title compound obtained as a tan solid in 40% yield using a process analogous to that of Example 5 with an intermediate prepared from 2,5-dibromofuran reacted with a commercially available boronic acid in manner analogous to the preparation of intermediate A. ¹H NMR (300.132 MHz, DMSO) 6 7.75-7.71 (m, 2H), 7.49-7.43 (m, 1H), 7.37-7.34 (m, 1H), 7.07 (d, J=3.5 Hz, 1H), 6.25 (d, J=3.4 Hz, 1H), 4.29 and 4.06 (d, J=9.6 Hz, 2H), 3.52-3.45 (m, 2H), 3.40-3.34 (m, 2H), 3.11-2.97 (m, 2H), 2.80-2.64 (m, 4H), 2.11 (s, 1H), 1.93-1.77 (m, 5H), 1.72-1.54 (m, 2H), 1.54-1.40 (m, 2H); MS m/z: 422 (M+H)+.

### EXAMPLE 13

### 5-(R)-1-Aza-bicyclo[2.2.2]oct-3-yl-3-{5-[3-(morpholine-4-carbonyl)-phenyl)-phenyl]-furan-2-yl}-oxazolidin-2-one

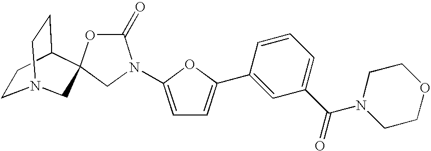

The title compound obtained as a tan solid in 77% yield using a process analogous to that of Example 10 with 2,5-dibromofuran and a commercially available boronic acid. ¹H NMR (300.132 MHz, DMSO) δ7.74 (d, J=8.8 Hz, 1H), 7.66 (s, 1H), 7.47 (t, J=7.7 Hz, 1H), 7.26 (d, J=7.9 Hz, 1H), 7.08 (d, J=2.6 Hz, 1H), 6.26 (d, J=3.5 Hz, 1H), 4.29 and 4.06 (AB, J=10.3 Hz, 2H), 3.69-3.32 (m, 8H), 3.11-2.97 (m, 2H), 2.77-2.64 (m, 4H), 2.14-2.07 (m, 1H), 1.89-1.76 (m, 1H), 1.74-1.55 (m, 2H), 1.53-1.38 (m, 1H); MS m/z: 438 (M+H)+.

### EXAMPLE 14

### (3R)-3′-{5-[4-(pyrrolidine-1-carbonyl pyridin-2-yl]-thiophene-2-yl}spiro(1-azabicyclo[2.2.2]octan-3,5′-oxazolidin-2′-one)

(a) 2-Bromo-4-(pyrrolidine-1-carbonyl pyridine

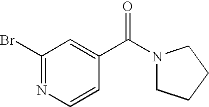

2-Bromo-2-pyridinecarboxylic acid (1.00 g, 5.0 mmol) was suspended in methylene chloride (15 mL) at 0° C. Oxalyl chloride (754 mg, 5.0 mmol) was added slowly into the solution at 0° C., and the mixture was then stirred at room temperature for 2 h. The reaction mixture was cooled to 0° C., then triethylamine (1.21 g, 9.9 mmol), 4-dimethylaminopyridine (121 mg, 0.99 mmol) followed by pyrrolidine (528 mg, 7.43 mmol) were added slowly. During the addition, the temperature was between 0 to 5° C. The reaction mixture was allowed to warm to room temperature, and stirred for another 1 h. The reaction mixture was then diluted with methylene chloride, washed with aqueous sodium bicarbonate, dried over MgSO₄, filtered, and evaporated. The residue was purified by flash chromatography using a gradient of ethyl acetate in hexane to give the sub-title compound as an oil, m/z 255,257 (MH⁺).

(b) 2-(Thiophene-2-yl)-4-(pyrrolidine-1-carbonyl)pyridine

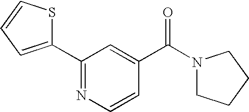

2-Bromo-4-(pyrrolidine-1-carbonyl)pyridine (609 mg, 2.39 mmol), 2-tri-n-butylstannyl thiophene (1.43 g, 3.82 mmol) and tetrakis(triphenylphosphine)palladium (0) (277 mg, 0.24 mmol) were heated under reflux in toluene (6 mL) overnight. The solution was allowed to cool, and was then filtered and evaporated. The residue was purified by flash chromatography using a gradient of ethyl acetate in hexane to give the title compound as a colorless solid (600 mg); m/z 259 (MH⁺).

(c) 2-(5-Bromothiophene-2-yl)-4-(pyrrolidine-1-carbonyl)pyridine

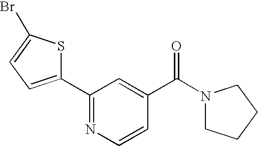

2-(Thiophene-2-yl)-4-(pyrrolidine-1-carbonyl)pyridine (579 mg, 2.24 mmol) was dissolved in dimethylformamide (2 mL). N-bromosuccinimide (479 mg, 2.69 mmol) was added into the solution, and the reaction mixture was stirred at room temperature for 1 h. The reaction mixture was diluted with chloroform, washed with saturated aqueous sodium bicarbonate, then dried over MgSO₄, filtered, and evaporated. The residue was purified by flash chromatography using a gradient of ethyl acetate in hexane to give the sub-title compound as a viscous oil (792mg); m/z 337, 339 (MH⁺).

(d) (3R)-3′-{5-[4-(pyrrolidine-1-carbonyl)pyridin-2-yl]thiophene-2-yl} spiro(1-azabicyclo[2.2.2]octan-3 ,5′-oxazolidin-2′-one)

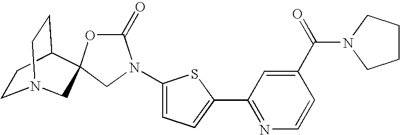

(3S)-Spiro(1-azabicyclo[2.2.2]octan-3,5′-oxazolidin-2′-one) (100 mg, 0.54 mmol), 2-(5-bromothiophene-2-yl)-4-(pyrrolidine-1-carbonyl)pyridine (208 mg, 0.61 mmol), cuprous iodide (514 mg, 2.70 mmol), ethylenediamine (162 mg, 2.70 mmol) and potassium carbonate (149 mg, 1.08 mmol) in 1,4-dioxane (5 mL) were stirred at 110° C. overnight. The solution was allowed to cool, and was then filtered, washed with aqueous ammonium hydroxide and evaporated. The residue was purified by flash chromatography using a gradient of ammoniated methanol in chloroform. The solid obtained from flash chromatography was further purified by reverse phase HPLC on a Polar RP C18 Column (250×30 mm, 4 μ micro) using a gradient of 5-45% acetonitrile/water (each solvent containing 0.1% formic acid as a buffer) as the eluant. The product-containing fractions were evaporated. The residue was taken up in saturated aqueous potassium carbonate, and extracted with chloroform to give the title compound as a pale solid (41 mg); in/z 439 (MH^{+).}

### EXAMPLE 15

### (3R)-3′-{5-[6-(pyrrolidine-1-carbonyl) pyridin-2-yl]thiophene-2-yl}spiro[1-azabicyclo[2.2.2]octan-3,5′-oxazolidin-2′-one]

(a) 2-Bromo-6-(pyrrolidine-1-carbonyl)-pyridine

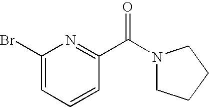

The sub-title compound was prepared by a method analogous to that described for the synthesis of 2-bromo-4-(pyrrolidine-1-carbonyl)pyridine from 6-bromopyridine-2-carboxylic acid and pyrrolidine, and was obtained as an oil; m/z 255, 257 (MH⁺).

(b) 2-(Thionhene-2-yl)-6-(pyrrolidine-1-carbonyl)pyridine

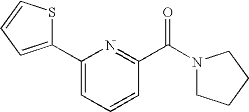

The sub-title compound was prepared by a method analogous to that described for the synthesis of 2-(thiophene-2-yl)-4-(pyrrolidine-1-carbonyl)pyridine from 2-bromo-6-(pyrrolidine-1-carbonyl)pyridine and 2-(tri-n-butylstannyl)thiophene, and was obtained as a gum; m/z 259 (MH⁺).

(c) 2-(5-Bromothiophene-2-yl)-6-(pyrrolidine-1-carbonyl)pyridine

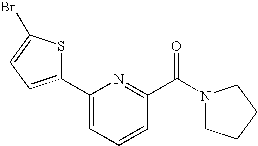

The sub-title compound was prepared by a method analog to that described for the synthesis of 2-(5-bromothiophene-2-yl)-4-(pyrrolidine-1-carbonyl)pyridine from 2-(thiophene-2-yl)-6-(pyrrolidine-1-carbonyl)pyridine, and was obtained as a gum; m/z 337 339 (MH⁺).

(d) (3R)-3′-{5-[6-(pyrrolidine-1-carbonyl)pyridin-2-yl]thiophene-2-yl}spiro[1-azabicyclo[2.2.2]octan-3,5′-oxazolidin-2′-one]

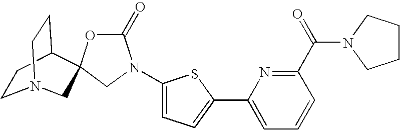

The title compound was prepared by a method analogous to that described for the synthesis of (3R)-3′-{5-[4-(pyrrolidine-1-carbonyl)pyridin-2-yl]thiophene-2-yl}spiro[1-azabicyclo[2.2.2]octan-3,5′-oxazolidin-2′-one] from (3S)-spiro[1-azabicyclo[2.2.2]octan-3,5′-oxazolidin-2′-one] and 2-(5-bromothiophene-2-yl)-6-(pyrrolidine-1-carbonyl)pyridine, and was obtained as a pale solid; m/z 439 (MH⁺).

### EXAMPLE 16

### (3R)-3′-{5-[4-(Morpholine-4-carbonyl)pyridin-2-yl]thiophene-2-yl}spirol[1azabicylo[2.2.2]octan-3,5′-oxazolidin-2′-one]

### 2-Bromo-4-(morpholine-4-carbonyl)pyridine

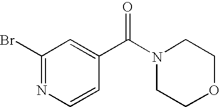

2-Bromopyridine-4-carboxylic acid (1.00 g, 4.96 mmol), 1-hydroxybenzotriazole hydrate (0.85 g, 5.56 mmol), 0-benzotriazol-1-yl-N,NN,N′-tetramethyluroniumtetrafluoroborate (1.81 g, 5.64 mmol), (N,N-dimethylamino)pyridine (20 mg), N,N-diisopropylethylamine (3.5 mL, 2.59 g, 20 mmol), and morpholine (0.87 ml, 0.87 g, 10 mmol) in dimethylformamide (15 ml) were stirred at room temperature overnight. The solution was evaporated and the residue was partitioned between chloroform and water. The organic extract was dried over MgSO₄, filtered, and evaporated. The residue was purified by flash chromatography on silica using a gradient of 0-100% ethyl acetate and hexane to give the sub-title compound as an oil; m/z 271, 273 (MH⁺).

(b) 2-(Thiophene-2-yl)-4-(4-morpholine-carbonyl)pyridine

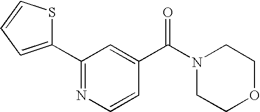

The sub-title compound was prepared by a method analogous to that described for the preparation of 2-(thiophene-2-yl)-4-(pyrrolidine-1-carbonyl)pyridine from 2-bromo-4-(morpholine-4-carbonyl)pyridine and 2-(tri-n-butylstannyl)thiophene, and was obtained as a light-yellow solid; m/z 275 (MH⁺).

(c) 2-(5-Bromo-thiophene-2-yl)-4-(morpholine-4-carbonyl)pyridine

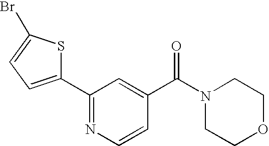

The sub-title compound was prepared by a method analogous to that described for the preparation of 2-(5-bromothiophene-2-yl)-4-(pyrrolidine-1-carbonyl)pyridine from 2-(thiophene-2-yl)-4-(4-morpholine-carbonyl)pyridine, and was obtained as a gum; m/z 353 355 (MH⁺).

(d) (3R)-3′-{5-[4-(morpholine-4-carbonyl)pyridin-2-yl]thiophene-2-yl} spiro[1-azabicyclo[2.2.2]octan-3,5′-oxazolidin-2′-one]

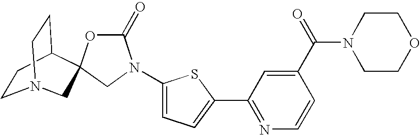

The title compound was prepared by a method analogous to that described for the preparation of (3R)-3′-{5-[4-(pyrrolidine-1-carbonyl)pyridin-2-yl]-thiophene-2-yl}spiro(1-azabicyclo[2.2.2]octan-3,5′-oxazolidin-2′-one) from (3S)-spiro[1-azabicyclo[2.2.2]octan-3,5′-oxazolidin-2′-one] and 2-(5-bromothiophene-2-yl)-4-(morpholine-4-carbonyl)pyridine, and was obtained as a yellow solid; m/z 455 (MH⁺).

### EXAMPLE 17

### (3R)-3′-{5-[6-(Morpholine-4-carbonyl)pyridin-2-yl]thiophene-2-yl}spiro[1-azabicyclo[2.2.2]octan-3,5′-oxazolidin-2′-one]

(a) 2-Bromo-6-(morpholine-4-carbonyl)pyridine

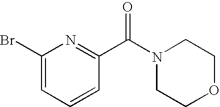

The sub-title compound was prepared by a method analogous to that described for the preparation of 2-bromo-4-(morpholine-4-carbonyl)pyridine from 6-bromopyridine-2- carboxylic acid and obtained as an oil; m/z 271, 273 (MH⁺).

(b) 2-(Thiophene-2-yl)-6-(morpholine-4-carbonyl)pyridine

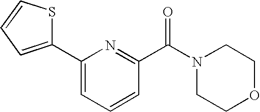

The sub-title compound was prepared by a method analogous to that described for the preparation of 2-(thiophene-2-yl)-4-(pyrrolidine-1-carbonyl)pyridine from 2-bromo-6-(morpholine-4-carbonyl)pyridine and 2-(tri-n-butylstannyl)thiophene, and was obtained as a light-yellow solid; m/z 275 (MH⁺).

(c) 2-(5-Bromothiophene-2-yl)-6-(4-morpholine-4-carbonyl)pyridine

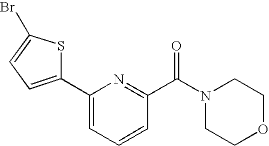

The sub-title compound was prepared by a method analogous to that described for the preparation of 2-(5-bromothiophene-2-yl)-4-(pyrrolidine-1-carbonyl)pyridine from 2-(thiophene-2-yl)-6-(morpholine-4-carbonyl)pyridine and was obtained as a gum; m/z 353 355 (MH⁺).

(d) (3R)-3′-{5-[6-(moroholine-4-carbonyl)pyridin-2-yl]thiophene-2-yl}spiro[1-azabicyclo[2.2.2]octan-3,5′-oxazolidin-2′-one]

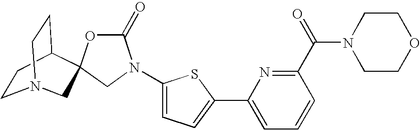

The title compound was prepared by a method analogous to that described for the preparation of (3R)-3′-{5-[4-(pyrrolidine-1-carbonyl)pyridin-2-yl]-thiophene-2-yl}spiro(1-azabicyclo[2.2.2]octan-3,5′-oxazolidin-2′-one) from (3S)-spiro[1-azabicyclo[2.2.2]octan-3,5′-oxazolidin-2′-one] and 2-(5-bromo-thiophene-2-yl)-6-(4-morpholine-carbonyl)-pyridine, and was obtained as a yellow solid; m/z 327 (MH⁺).

### EXAMPLE 18

### (3R)-3′-[5-(4-Pyridyl)oxazol-2-yl]spiro[1-azabicyclo[2.2.2]octan-3,5′-oxazolidin-2′-one]

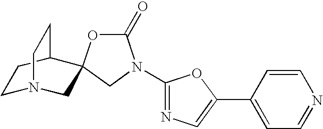

(a) 5-(4-Pyridyl)oxazole

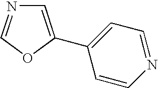

4-Pyridinecarboxaldehyde (1.12 g, 10.5 mmol), tosylmethyl isocyanide (2.05 g, 10.5 mmol) and potassium carbonate (1.45 g, 10.5 mmol) were heated under reflux in methanol (30 mL) at 70 ° C. for 3 h. The solution was allowed to cool, and then evaporated. The residue was dissolved in chloroform, washed with saturated aqueous potassium carbonate, dried (MgSO₄), filtered, and evaporated. The residue was purified by flash chromatography using a gradient of ethyl acetate in hexane to give the sub-title compound as a light yellow solid (1.10 g); m/z 147 (MH⁺)).

(b) 2-Iodo-5-(4-pyridyl)oxazole

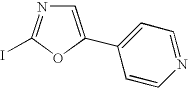

5-(4-Pyridyl)oxazole (960 mg, 6.6 mmol) was dissolved in anhydrous ether (80 mL), and cooled to −78° C. Lithium bis(trimethylsilyl)amide (1 M in THF, 8.54 mL, 8.54 mmol) was added slowly into the resulting suspension, and the reaction mixture was then stirred for 1 h at −78° C. A solution of 1,2-diiodoethane (2.40 g, 8.54 mmol) in anhydrous ether (20 mL) was then added slowly into the reaction mixture at −78° C. After the addition was complete, the reaction mixture was allowed to warm to room temperature, and stirred for another 1 h. The reaction mixture was quenched with wet ether followed by saturated aqueous potassium carbonate aqueous solution and then extracted with ether. The combined organic layer extracts were washed with aqueous sodium thiosulfate, dried (MgSO₄), filtered, and evaporated. The residue was purified by flash chromatography using a gradient of ethyl acetate in hexane to give the sub-title compound as a solid m/z 273 (MH⁺).

(c) (3R)-3′-[5-(4-Pyridyl)oxazol-2-yl]spiro[1-azabicyclo[2.2.2]octan-3,5′-oxazolidin-2′-one]

Sodium hydride (66 mg, 1.65 mmol) was added into a solution of (3S)-spiro[1-azabicyclo[2.2.2]octan-3,5′-oxazolidin-2′-one] (200 mg, 1.10 mmol) in dimethylformamide (2 mL) at room temperature, and the reaction mixture was then heated to 50° C. for 30 min. 2-Iodo-5-(4-pyridyl)oxazole (449 mg, 1.65 mmol) in dimethylformamide (3 mL) was added into the reaction mixture which was then stirred at 50° C. overnight. The mixture was allowed to cool, then quenched with saturated aqueous ammonium chloride, diluted with a large volume of chloroform, washed with saturated aqueous potassium carbonate, dried (MgSO₄), filtered, and evaporated. The residue was purified by flash chromatography using a gradient of ammoniated methanol in chloroform to give a yellow-brown solid. The solid obtained from flash chromatography was further purified by reverse phase HPLC on a Polar RP C18 Column (250×30 mm, 4 u micro) using a gradient of 5-45% acetonitrile/water (each solvent containing 0.1% formic acid as a buffer) as the eluant. The product-containing fractions were evaporated and the residue was washed with saturated aqueous potassium carbonate, then extracted with chloroform, dried (MgSO₄), filtered, and evaporated to give the title compound (71 mg) as a solid; m/z 327 (MH⁺).

### EXAMPLE 19

### (3R)-3′-[5-(3-Pyridyl)oxazol-2-yl]spiro[1-azabicyclo[2.2.2]octan-3,5′- 15 oxazolidin-2′-one]

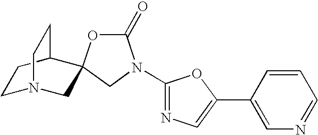

(a) 2-Iodo-5-(3-pyridyl)oxazole

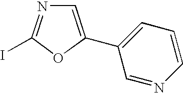

The sub-title compound was prepared by a method analogous to that described for the preparation of 2-iodo-5-(4-pyridyl)oxazole from 5-(3-pyridyl)oxazole and obtained as a yellow solid; m/e 273 (MH⁺).

(b) (3R)-3′-[5-(3-Pyridyl)oxazol-2-yl]spiro[1-azabicyclo[2.2.2]octan-3,5′-oxazolidin-2′-one]

The title compound was prepared by a method analogous to that described for the preparation of (3R)-3′-[5-(4-Pyridyl)oxazol-2-yl]spiro[1-azabicyclo[2.2.2]octan-3,5′-25 oxazolidin-2′-one] from (³S)-spiro[1-azabicyclo[2.2.2]octan-3,5′-oxazolidin-2′-one] and 2-iodo-5-(3-pyridyl)-oxazole, and was obtained as a light-yellow solid; m/z 327 (MH⁺).

### EXAMPLE 20

### (3R)-3′-(5-(2-Pyridyl)oxazol-2-yl)spiro[1-azabicyclo[2.2.2]octan-3,5′-oxazolidin-2′-one]

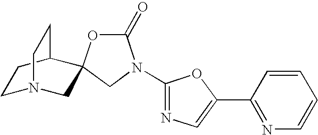

(a) 5-(2-Pyridyl)oxazole

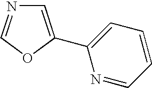

The sub-title compound was prepared by a method analogous to that described for the preparation of 5-(4-pyridyl)oxazole from 2-pyridinecarboxaldehyde and obtained as a light yellow oil; m/z 147 (MH⁺).

(b) 2-Iodo-5-(2-pyridyl)oxazole

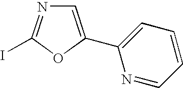

The subtitle compound was prepared by a method analogous to that described for the preparation of 2-iodo-5-(4-pyridyl)oxazole from 5-(2-pyridyl)oxazole and obtained as a yellow solid; m/z 273 (MH⁺).

(c) (3R)-3′-[5-(2-Pyridyl)oxazol-2-yl]spiro[1-azabicyclo[2.2.2]octan-3,5′-oxazolidin-2′-one]

The title compound was prepared by a method analog to that described for the preparation of (3R)-3′-[5-(4-Pyridyl)oxazol-2-yl]spiro[1-azabicyclo[2.2.2]octan-3,5′-oxazolidin-2′-one] from (3S)-spiro[l-azabicyclo[2.2.2]octan-3,5′-oxazolidin-2′-one] and 2-iodo-5-(2-pyridyl)oxazole. The hydrochloride salt of the title compound was obtained as a solid; m/z 327 (MH⁺).

### EXAMPLE 21

### (3R)-3′-(5-(2-Pyridyl)thiazol-2-yl)spiro[1-azabicyclo[2.2.2]octan-3,5′-oxazolidin-2′-one]

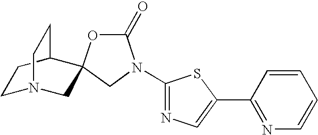

A mixture containing (3R)-3′-(5-bromothiazol-2-yl)spiro[1-azabicyclo[2.2.2]octan-3,5¹-oxazolidin-2′-one] (200 mg, 0.58 mmol), ³-tri-n-butylstannylpyridine (755 mg, 1.74 mmol), tetrakis(triphenylphosphine)palladium(0) (67 mg, 0.06 mmol), in toluene (5 mL), was heated at 110° C. overnight. The reaction mixture was filtered and then purified by column chromatography using 0-5% ammoniated methanol in chloroform as the eluant, followed by HPLC using a gradient of water and acetonitrile containing 0.1% trifluoroacetic acid as the eluant. The product-containing fractions were evaporated and then partitioned between aqueous potassium carbonate solution and chloroform. The organic layer was separated, dried (MgSO₄), filtered and evaporated to give the title compound (76 mg) as a solid; m/z 343 (MH⁺).

### EXAMPLE 22

### (3R)-3′-(5-(3-Pyridyl)thiazol-2-yl)spiro[1-azabicyclo[2.2.2]octan-3,5′-oxazolidin-2′-one]

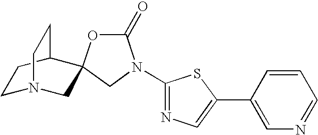

The title compound was prepared by a method analogous to that described in Example 1 from (3R)-3′-(5-bromothiazol-2-yl)spiro[1-azabicyclo[2.2.2]octan-3,5′-oxazolidin-2′-one] and 3-tri-n-butylstannylpyridine. The title compound (76 mg) was obtained as a solid; m/z 343 (MH⁺).

1. A compound according to Formula I: 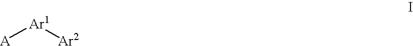
wherein:
A is 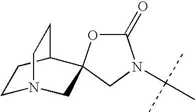
Ar¹ and Ar2 are independently a 5- or 6-membered aromatic or heteroaromatic moiety having 0, 1 or 2 nitrogen atoms, 0 or 1 oxygen atoms, and 0 or 1 sulfur atoms;
wherein Ar¹ is unsubstituted or has 1, 2 or 3 substituents independently selected from -C₁-C₆alkyl, -C₂-C₆alkenyl, -C₂-C₆alkynyl, —CN, —NO₂, —CF₃, —S(O)ₙR¹ where n is 0, 1 or 2,—NR¹R², —CH₂—NR¹R², —OR², —CH₂OR² or —CO₂R², and
Ar² is unsubstituted or has 1, 2 or 3 substituents selected from —C(═O)—NR¹R² or —NR¹—C(═O)—R²;
where at each occurrence R¹ and R² are independently selected from hydrogen or -C₁-C₆alkyl, or —NR¹R² in combination is —(CH₂)ⱼG(CH₂)ₖ- wherein G is a bond, oxygen, sulfur or NR³ where R³ is selected from hydrogen, C₁₋₆alkyl, aryl or heteroaryl. j is 2, 3 or 4. and k is 0, 1 or 2, or a stereoisomer, enantiomer, in vivo-hydrolysable precursor and pharmaceutically-acceptable salt thereof.

2. A compound according to claim 1, wherein:
Ar¹ is a 5-membered heteroaromatic ring having 1 oxygen atom or 1 sulfur atom and
Ar² is a 6-membered aromatic ring or a heteroaromatic ring having 0, 1 or 2 nitrogen atoms, 0 or 1 oxygen atoms, and 0 or 1 sulfur atoms.
or a stereoisomer, enantiomer, in vivo-hydrolysable precursor and pharmaceutically-acceptable salt thereof.

3. A compound according to claim 1, wherein:
Ar¹ is an unsubstituted 5-membered heteroaromatic ring having 1 oxygen atom or 1 sulfur atom;
Ar² is a 6-membered aromatic ring or a heteroaromatic ring having 0, 1 or 2 nitrogen atoms, 0 or 1 oxygen atoms, and 0 or 1 sulfur atoms substituted with 1 or 2 substituents selected from —C(═O)—NR¹R² or —NR¹—C(═O)—R² where at each occurrence R¹ and R² are independently selected from hydrogen or -C₁-C₆alkyl, or —NR¹R² in combination is —(CH₂)ⱼG(CH₂)ₖ- wherein G is a bond, oxygen, sulfur or NR³ where R³ is selected from hydrogen, C ₁₆alkyl, aryl or heteroaryl; j is 2, 3 or 4, and k is 0, 1 or2,
or a stereoisomer, enantiomer, in vivo-hydrolysable precursor and pharmaceutically-acceptable salt thereof.

4. A compound according to claim 1, wherein:
Ar¹ is an unsubstituted 5-membered heteroaromatic ring having 1 oxygen atom or 1 sulfur atom;
Ar² is a 6-membered aromatic ring or a heteroaromatic ring having 0, 1 or 2 nitrogen atoms, 0 or 1 oxygen atoms, and 0 or 1 sulfur atoms substituted with 1 substituent selected from —C(═O)—NR¹R² or —NR¹—C(═O)—R² where at each occurrence R¹ and R² are independently selected from hydrogen or -Cl-C₆alkyl, or —NR¹R² in combination is —(CH₂)ⱼG(CH₂)ₖ- wherein G is a bond, oxygen, sulfur or NR³ where R³ is selected from hydrogen, C₁₋₆alkyl, aryl or heteroaryl; j is 2, 3 or 4, and k is 0, 1 or 2, or a stereoisomer, enantiomer, in vivo-hydrolysable precursor and pharmaceutically-acceptable salt thereof.

5. A compound according to claim 1, selected from:
4-[5-((R)-5-1-Aza-bicyclo[2.2.2]oct-3-yl-2-oxo-oxazolidin-3-yl)-thiophen-2-yl]-N,N-dimethyl-benzamide;
3-[5-((R)-5-1-Aza-bicyclo[2.2.2]oct-3-yl-2-oxo-oxazolidin-3-yl)-thiophen-2-yl]-N,N-dimethyl-benzamide;
N-{3-[5-((R)-5-1-Aza-bicyclo[2.2.2]oct-3-yl-2-oxo-oxazolidin-3-yl)-thiophen-2-yl]-phenyl}-acetamide;
N-{4-[5-((R)-5-1-Aza-bicyclo[2.2.2]oct-3-yl-2-oxo-oxazolidin-3-yl)-thiophen-2-yl]-phenyl}-acetamide;
5-(R)-1-Aza-bicyclo[2.2.2]oct-3-yl-3-{5-[3-(pyrrolidine-1-carbonyl)-phenyl]-thiophen-2-yl}-oxazolidin-2-one;
N-{3-[5-((R)-5-1-Aza-bicyclo[2.2.2]oct-3-yl-2-oxo-oxazolidin-3-yl)-thiophen-2-yl]-phenyl}-propionamide;
N-{3-[5-((R)-5-1-Aza-bicyclo[2.2.2]oct-3-yl-2-oxo-oxazolidin-3-yl)-thiophen-2-yl]-phenyl}-N-methyl-acetamide;
N-{3-[5-((R)-5-1-Aza-bicyclo[2.2.2]oct-3-yl-2-oxo-oxazolidin-3-yl)-thiophen-2-yl]-phenyl}-N-methyl-propionamide;
N-{4-[5-((R)-5-1-Aza-bicyclo[2.2.2]oct-3-yl-2-oxo-oxazolidin-3-yl)-thiophen-2-yl]-phenyl}-N-methyl-acetamide;
5-(R)-1-Aza-bicyclo[2.2.2]oct-3-yl-3-{5-[3-(morpholine-4-carbonyl)-phenyl]-thiophen-2-yl}-oxazolidin-2-one;
N-{3-[5-((R)-5-1-Aza-bicyclo[2.2.2]oct-3-yl-2-oxo-oxazolidin-3-yl)-furan-2-yl]-phenyl}-propionamide;
5-(R)-1-Aza-bicyclo[2.2.2]oct-3-yl-3-{5-[3-(pyrrolidine-1-carbonyl)-phenyl]-furan-2-yl}-oxazolidin-2-one;
5-(R)-1-Aza-bicyclo[2.2.2]oct-3-yl-3-{5-[3-(morpholine-4-carbonyl)-phenyl)-phenyl]-furan-2-yl}-oxazolidin-2-one;
(3R)-3′-{5-[4-(pyrrolidine-1-carbonyl)pyridin-2-yl]-thiophene-2-yl}spiro(1-azabicyclo[2.2.2]octan-3,5′-oxazolidin-2′-one);
(3R)-3′-{5-[6-(pyrrolidine-1-carbonyl)pyridin-2-yl]thiophene-2-yl} spiro[ 1-azabicyclo[2.2.2]octan-3 ,5′-oxazolidin-2′-one];
(3R)-3′-{5-[4-(Morpholine-4-carbonyl)pyridin-2-yl]thiophene-2-yl} spiro[ 1-azabicyclo[2.2.2]octan-3,5′-oxazolidin-2′-one];
(3R)-3′-{5-[6-(Morpholine4-carbonyl)pyridin-2-yl]thiophene-2-yl} spiro[ 1-azabicyclo[2.2.2]octan-3,5′-oxazolidin-2′-one];
(3R)-3′-[5-(4-Pyridyl)oxazol-2-yl]spiro[1-azabicyclo[2.2.2]octan-3,5′-oxazolidin-2′-one];
(3R)-3′-[5-(3-Pyridyl)oxazol-2-yl]spiro[1-azabicyclo[2.2.2]octan-3,5′-oxazolidin-2′-one];
(3R)-3′-(5-(2-Pyridyl)oxazol-2-yl )spiro[1-azabicyclo[2.2.2]octan-3,5′-oxazolidin-2′-one];
(3R)-3′-(5-(2-Pyridyl)thiazol-2-yl)spiro[1-azabicyclo[2.2.2]octan-3,5′-oxazolidin-2′-one], and
(3R)-3′-(5-(3-Pyridyl)thiazol-2-yl)spiro[1-azabicyclo[2.2.2]octan-3,5′-oxazolidin-2′-one],
or a stereoisomer, enantiomer, in vivo-hydrolysable precursor and pharmaceutically-acceptable salt thereof.

6. A compound according to Formula Ia: 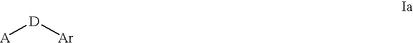
wherein:
A is 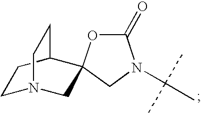
D is a 5-membered heteroaromatic moiety having one nitrogen atom and one oxygen atom or a heteroaromatic moiety having one nitrogen atom and one sulfur atom, and
wherein Ar is unsubstituted or has 1, 2 or 3 substituents independently selected from -C₁-C₆alkyl, -C₂-C₆alkenyl, -C₂-C₆alkynyl, —CN, —NO₂, —CF₃, —S(O)ₙR¹ where n is 0, 1 or 2,—NR¹R², —CH₂—NR¹R², —OR², —CH₂OR² or —CO₂R², where at each occurrence R¹ and R² are independently selected from hydrogen or -C₁-C₆alkyl, or —NR¹R² in combination is —(CH₂)ⱼG(CH₂)ₖ- wherein G is a bond, oxygen, sulfur or NR³ where R³ is selected from hydrogen, C₁₋₆alkyl, aryl or heteroaryl; j is 2, 3 or 4, and k is 0, 1 or 2;
or a stereoisomer, enantiomer, in vivo-hydrolysable precursor and pharmaceutically-acceptable salt thereof.

7. A compound according to claim 6, wherein:
wherein:
D is selected from moieties according to formulae II, III, IV and V 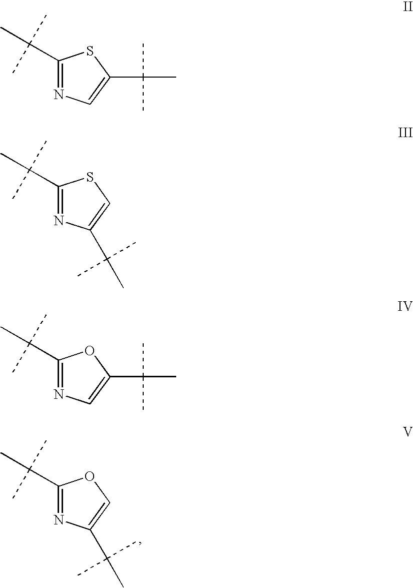
or a stereoisomer, enantiomer, in vivo-hydrolysable precursor and pharmaceutically-acceptable salt thereof.

8. A compound according to claim 6, wherein:
Ar is a 6-membered aromatic ring or a heteroaromatic ring having 0, 1 or 2 nitrogen atoms, substituted with 1 or 2 substituents selected from —C(═O)—NR¹R² or —NR¹—C(═O)—R² where at each occurrence R¹ and R² are independently selected from hydrogen or -C₁-C₆alkyl, or —NR¹R² in combination is —(CH₂)ⱼG(CH₂)ₖ- wherein G is a bond, oxygen, sulfur or NRW where R³ is selected from hydrogen, C₁₋₆alkyl, aryl or heteroaryl; j is 2, 3 or 4, and k is 0, 1 or 2, or a stereoisomer, enantiomer, in vivo-hydrolysable precursor and pharmaceutically-acceptable salt thereof.

9. A method of treatment or prophylaxis of a disease or condition in which activation of the α7 nicotinic receptor is beneficial which method comprises administering a therapeutically-effective amount of a compound according to claim 1 to a subject suffering from said disease or condition.

10. The method of claim 9, wherein said disease or condition is anxiety, schizophrenia, mania or manic depression.

11. A method of treatment or prophylaxis of neurological disorders, psychotic disorders or intellectual impairment disorders, which comprises administering a therapeutically effective amount of a compound according to claim 1.

12. The method of claim 11, wherein said disorder is Alzheimer's disease, learning deficit, cognition deficit, attention deficit, memory loss, Attention Deficit Hyperactivity Disorder, Parkinson's disease, Huntington's disease, Tourette's syndrome, neurodegenerative disorders in which there is loss of cholinergic synapses, jetlag, nicotine addiction, craving, pain, or ulcerative colitis.

13. A method for inducing the cessation of smoking comprising administering an effective amount of a compound according to claim 1.

14. A pharmaceutical composition comprising a compound according to claim 1 and a pharmaceutically-acceptable diluent, lubricant or carrier.

15. A method of treatment or prophylaxis of a disease or condition in which activation of the α7 nicotinic receptor is beneficial which method comprises administering a therapeutically-effective amount of a pharmaceutical composition according to claim 1 to a subject suffering from said disease or condition.

16. The method of claim 15, wherein said disease or condition is anxiety, schizophrenia, mania or manic depression.

17. A method of treatment or prophylaxis of neurological disorders, psychotic disorders or intellectual impairment disorders, which comprises administering a therapeutically effective amount of a pharmaceutical composition according to claim 14.

18. The method of claim 17, wherein said disorder is Alzheimer's disease, learning deficit, cognition deficit, attention deficit, memory loss, Attention Deficit Hyperactivity Disorder, Parkinson's disease, Huntington's disease, Tourette's syndrome, neurodegenerative disorders in which there is loss of cholinergic synapses, jetlag, nicotine addiction, craving, pain, or ulcerative colitis.

19. A method for inducing the cessation of smoking comprising administering an effective amount of a pharmaceutical composition according to claim 14.

20. The use of a compound according to claim 1, an enantiomer thereof or a pharmaceutically-acceptable salt thereof, in the manufacture of a medicament for the treatment or prophylaxis of human diseases or conditions in which activation of the a7 nicotinic receptor is beneficial selected from neurological disorders, psychotic disorders, intellectual impairment disorders, Alzheimer's disease, learning deficit, cognition deficit, attention deficit, memory loss, Attention Deficit Hyperactivity Disorder, anxiety, schizophrenia, mania or manic depression, Parkinson's disease, Huntington's disease, Tourette's syndrome, or neurodegenerative disorders in which there is loss of cholinergic synapses.

21. The use of a compound according to claim 1, in the manufacture of a medicament for the treatment or prophylaxis of jetlag, pain, or ulcerative colitis or to facilitate the cessation of smoking or the treatment of nicotine addiction or craving including that resulting from exposure to products containing nicotine.
